(19) United States
(12) Patent Application Publication
(10) Pub. No.: US 2022/0331742 A1
(43) Pub. Date: Oct. 20, 2022

(54) SILICA ANTISCALANT COMPOSITION AND METHOD FOR SILICA SCALING INHIBITION IN MEMBRANE APPLICATIONS

(71) Applicant: BL Technologies, Inc., Minnetonka, MN (US)

(72) Inventors: Xingpeng ZHANG, Singapore (SG); Dongying YE, Singapore (SG); Jeffrey MELZER, Trevose, PA (US); Caroline SUI, Trevose, PA (US); Qiantao CAI, Singapore (SG); Monica SHARMA, Bangalore Karnataka (IN)

(21) Appl. No.: 17/640,521

(22) PCT Filed: Aug. 18, 2020
(86) PCT No.: PCT/US2020/046750

§ 371 Date: Mar. 4, 2022

Related U.S. Application Data
(60) Provisional application No. 62/896,939, filed on Sep. 6, 2019.

(51) Int. Cl. B01D 65/08; B01D 61/04; C02F 1/44; C02F 5/14

(52) U.S. Cl. CPC B01D 65/08; B01D 61/04; C02F 1/441; C02F 1/442; C02F 5/14; B01D 2321/162

(57) ABSTRACT

An antiscalant composition, the composition having a silica inhibitor composition, and a dispersant composition. A method for inhibiting scale formation in a membrane system, the method providing an antiscalant composition, the antiscalant composition having a silica inhibitor and a dispersant, and adding the antiscalant composition to an aqueous stream of an aqueous system.

### CROSS REFERENCE TO RELATED APPLICATIONS

This application claims the priority benefit of U.S. Provisional Patent Application Ser. No. 62/896,939 filed Sep. 6, 2019, the entirety of which is incorporated herein by reference.

### FIELD OF THE INVENTION

The disclosed technology generally provides for a composition and method for silica scaling inhibition in membrane applications, and more specifically, a membrane silica antiscalant and method for silica scaling inhibition in high silica water membrane applications.

### BACKGROUND OF THE INVENTION

In the membrane desalination industry, operators generally run membrane systems at a higher recovery rate and reduce concentrate disposal in order to save on operating costs. However, this objective is quite challenging in waters with elevated concentration of silica. With high concentrations of silica, build-up of silica scaling or silica deposits lead to lower productivity, poor product quality, unscheduled downtime, and frequent membrane clean-in-place (CIP) operations. Additionally, once silica scaling is formed on a membrane surface, it is nearly impossible to remove.

Silica scaling in membrane systems is very complicated and affected by many factors (e.g. silica level, pH value, temperature, other metal ions, system operating conditions). Among them, silica level and pH value are two most crucial factors. Generally, there are two approaches to improve system recovery and reduce concentrate disposal for high silica water treatment in membrane applications. The first approach is to adjust feed pH by acid addition. However, acid addition requires an additional feed/metering pump and handling of concentrated acid (a potential safety issue) body/skin contact, inhalation of vapors and etc. The second approach would be to dose a highly effective silica antiscalant.

Thus, what is needed in the art is a composition and method for silica scaling inhibition in high silica water membrane applications.

### SUMMARY OF THE INVENTION

The disclosed technology generally provides for a composition and method for silica scaling inhibition in membrane applications, and more specifically, a membrane silica antiscalant and method for silica scaling inhibition in high silica water membrane applications.

In one aspect of the disclosed technology, an antiscalant composition is provided. The antiscalant composition comprising a silica inhibitor composition; and a dispersant composition.

In some embodiments, the silica inhibitor composition comprises an organophosphoric acid, a phosphonate-based compound, or a carboxylic sulphonated copolymer. In some embodiments, the organophosphoric acid is 1-hydroxyethylidine-1,1-diphosphonic acid. In some embodiments, the silica inhibitor is present at a concentration of about 5-40% actives.

In some embodiments, the dispersant composition comprises a sulphonated acrylic acid polymer. In some embodiments, the sulphonated acrylic acid polymer comprises repeat units characterized by the formula

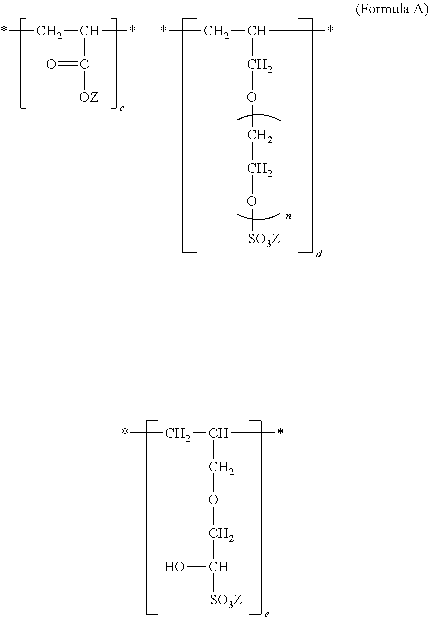

wherein n ranges from about 1-100; and Z is H, Na, K, Ca or NH₄.

In some embodiments, n is about 1-20. In some embodiments, Z may be the same or different in c, d and e. In some embodiments, the mole ratio of c:d:e ranges from about 20:10:1 to 1:1:20.

In some embodiments, the molecular weight of the sulphonated acrylic acid polymer ranges from about 10,000 to about 30,000. In some embodiments, the concentration ratio of the silica inhibitor composition to the dispersant composition is about 1:2. In some embodiments, the concentration ratio of the silica inhibitor composition to the dispersant composition is about 1:1.6. In some embodiments, the silica inhibitor and the dispersant composition are blended together.

In yet another aspect of the disclosed technology, an antiscalant composition is provided. The antiscalant composition comprising a blend of (i) a silica inhibitor composition, wherein the silica inhibitor composition comprises 1-hydroxyethylidine-1,1-diphosphonic acid; and (ii) a sulphonated/sulfated acrylic acid polymer or terpolymer.

In some embodiments, the sulphonated acrylic acid polymer or terpolymer comprises repeat units characterized by the formula

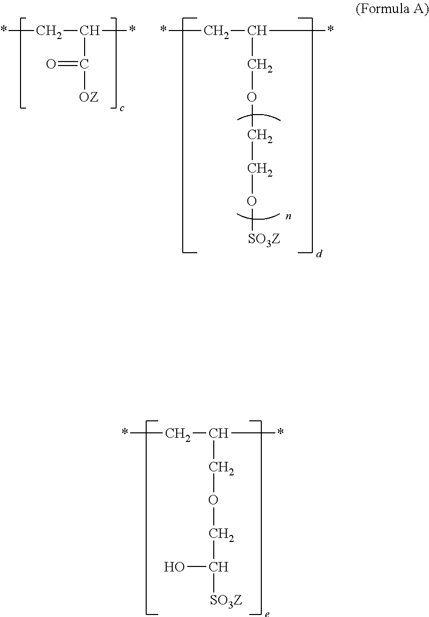

wherein n ranges from about 1-100; and Z is H, Na, K, Ca or NH₄.

In some embodiments, the silica inhibitor and the dispersant composition are blended together at about 25° C.

In yet another aspect of the disclosed technology, a method for inhibiting scale formation in a membrane system is provided. The method comprises providing an antiscalant composition, the antiscalant composition comprising a silica inhibitor and a dispersant; and adding the antiscalant composition to an aqueous stream of an aqueous system.

In some embodiments, the silica inhibitor comprises an organophosphoric acid, a phosphonate-based compound, or a carboxylic sulphonated copolymer. In some embodiments, the dispersant is a sulphonated acrylic acid polymer or terpolymer. In some embodiments, the antiscalant composition is a blend of the silica inhibitor and the dispersant.

In some embodiments, the silica inhibitor comprises 1-hydroxyethylidine-1,1-diphosphonic acid, and the dispersant is a sulphonated/sulfated acrylic acid polymer or terpolymer. In some embodiments, the sulphonated acrylic acid polymer or terpolymer comprises repeat units characterized by the formula

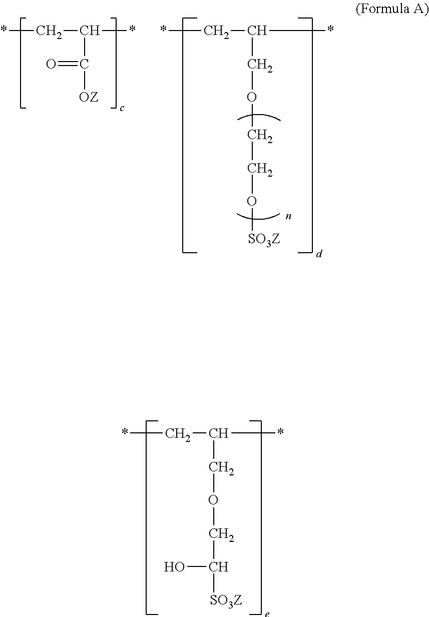

wherein n ranges from about 1-100; and Z is H, Na, K, Ca or NH₄.

In some embodiments, the aqueous stream comprises a silica content of at least 300 ppm. In some embodiments, the aqueous stream comprises a silica content of about 300 ppm to about 350 ppm. In some embodiments, the aqueous stream comprises a pH of at least 7. In some embodiments, the aqueous stream comprises a pH of about 7.5. In some embodiments, the aqueous stream has a pH of about 7.5 and a silica content of at least 300 ppm.

In some embodiments, the aqueous system comprises a reverse osmosis membrane or nanofiltration membrane. In some embodiments, the antiscalant composition is added to the aqueous stream in an amount of about 1 ppm to about 100 ppm. In some embodiments, the antiscalant composition is added to the aqueous stream in an amount of about 3 ppm to about 30 ppm.

### BRIEF DESCRIPTION OF SEVERAL VIEWS OF THE DRAWINGS

These and other features of the disclosed technology, and the advantages, are illustrated specifically in embodiments now to be described, by way of example, with reference to the accompanying diagrammatic drawings, in which:

FIG. 1 is a graph providing results of an illustrative embodiment of the disclosed technology;

FIG. 2 is a graph providing results of an illustrative embodiment of the disclosed technology;

FIG. 3 is a graph providing results of an illustrative embodiment of the disclosed technology;

FIG. 4 is a graph providing results of an illustrative embodiment of the disclosed technology; and

FIGS. 5A-5D provide results of an illustrative embodiment of the disclosed technology.

### DETAILED DESCRIPTION OF EXEMPLARY EMBODIMENTS

The disclosed technology generally provides for a composition and method for silica scaling inhibition in membrane applications, and more specifically, a membrane silica antiscalant and method for silica scaling inhibition in high silica water membrane applications.

The term “antiscalant” as used herein refers to a composition/formulation that inhibits (reduces) the formation of silica scale and/or the size and/or shape of solid silica particles.

It was surprisingly discovered that a blend of a silica inhibitor and a dispersant composition demonstrates a synergistic effect in silica scaling control. The membrane silica antiscalant composition as described herein was shown to be effective in treating feed streams containing high silica in membrane applications, such as reverse osmosis (RO) or nanofiltration (NF) systems under given process conditions. The membrane silica antiscalant composition allows for plant operation with concentrate silica levels of over 300 ppm, which exhibits a synergistic effect of threshold silica scaling inhibition and particle dispersion to extend membrane system recovery and lower operating cost.

In one aspect of the disclosed technology, an antiscalant composition is provided. The antiscalant composition comprises a silica inhibitor composition, and a dispersant composition. It was determined that a blend of the silica inhibitor composition and the dispersant composition as disclosed herein provides an effective treatment for handling high silica water treatment in membrane applications.

Generally, dissolved silica in feed water will be concentrated to a couple of times higher in RO or NF systems. This leads to silica polymerization, which grows into a large molecule or forms colloidal silica and/or particles, where such silica polymerization will be accelerated with the increasing silica level and pH value. It was surprisingly discovered that the blend of the silica inhibitor and dispersant composition allows for the silica antiscalant composition as described herein to postpone silica polymerization and keeps silica particles suspended in a stream from precipitating onto membrane surfaces.

Additionally, the disclosed silica antiscalant composition is non-toxic to the environment (i.e. “environmentally-friendly”), and eliminates the need for acid handling, because it allows for the treatment of high silica water without acid addition. The silica antiscalant composition as described herein avoids the increase of total dissolved solids (or TDS) of water resulting from the extra acid addition, which reduces energy consumption in water desalination and lower operating costs.

In some embodiments, the silica inhibitor composition comprises an organophosphoric acid, a phosphonate-based compound, or a carboxylic sulphonated copolymer. It is believed that the specific anionic groups of the silica inhibitor composition can interact with the cations in the feed solution to inhibit crystalline mineral salts precipitation, and reduce the opportunities of co-precipitating with silica colloid or particles, and thus aids in reducing silica scaling formation and minimizes the harmful impact on membrane performance.

In some embodiments, the organophosphoric acid is 1-hydroxyethylidine-1,1-diphosphonic acid (HEDP). In some embodiments, the silica inhibitor of the present technology will not only inhibit silica polymerization, but also effectively prevents calcium carbonate precipitation. In some embodiments, the silica inhibitor is present at a concentration of about 5-40% actives.

In some embodiments, the dispersant composition comprises a sulphonated acrylic acid polymer. In some embodiments, the sulphonated acrylic acid polymer comprises repeat units characterized by the formula

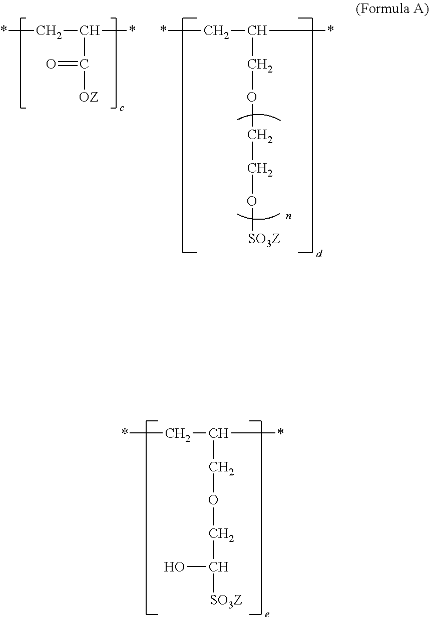

wherein n ranges from about 1-100; and Z is H, Na, K, Ca or NH₄.

In some embodiments, n is about 1-20.In other embodiments, n in about 10-20. In some embodiments, Z may be the same or different in c, d and e. In some embodiments, the mole ratio of c:d:e ranges from about 20:10:1 to 1:1:20.

In some embodiments, the sulphonated acrylic acid copolymer, terpolymer, or the sulphonated acrylic acid polymer comprising repeat units characterized by Formula A as described herein imparts a negative charge onto suspended silica particles present in a feed stream, which avoids agglomeration due to enhanced electrostatic repulsion and steric hinderance.

In some embodiments, the molecular weight of the sulphonated acrylic acid polymer ranges from about 10,000 to about 30,000. In other embodiments, the sulphonated acrylic acid polymer ranges from about 12,000 to about 25,000.

In some embodiments, the concentration ratio of the silica inhibitor composition to the dispersant composition is about 1:2. In some embodiments, the concentration ratio of the silica inhibitor composition to the dispersant composition is about 1:1.6.

In some embodiments, the silica inhibitor composition is about 5-25 wt. % and the dispersant composition is about 10-40 wt. % of the total antiscalant composition.

In some embodiments, the silica inhibitor and the dispersant composition are blended together. In some embodiments, the silica inhibitor and the dispersant composition are blended together at room temperature. In other embodiments, the silica inhibitor and the dispersant composition are blended together at about 25° C. It should be understood that blending may be provided by any conventional blending techniques sufficient for the purposes described herein. For example, but not limited to, conventional blending techniques may comprise flat-plate baffles, pitched-blade impellers, and/or a rushton turbine.

In some embodiments, the disclosed silica antiscalant composition may further include a phosphonate-based inhibitor. For example, but not limited to, diethylenetriamine penta(methylene phosphonic acid) (DTPMP), aminotris(methylenephosphonic acid) (ATMP), hexanediaminetetra(methylenephosphonic acid) (HDTMP), or the like. The presence of a phosphonated-based inhibitor may be necessary if/when the feed water or aqueous stream comprises a high CaCO3 precipitation potential. In such instances, the disclosed antiscalant composition can include the addition of a CaCO3 inhibitor.

In a specific embodiment, the antiscalant composition comprises a blend of (i) a silica inhibitor composition, wherein the silica inhibitor composition comprises 1-hydroxyethylidine-1,1-diphosphonic acid (HEDP), and (ii) a sulphonated/sulfated acrylic acid copolymer, terpolymer, or sulphonated acrylic acid polymer comprising repeat units characterized by Formula A.

In yet another aspect of the disclosed technology, a method for inhibiting scale formation in a membrane system is provided. The method as described herein does not include acid addition (i.e. allows for the treatment of high silica water without the need for acid addition as conventionally used). The method was shown to provide a synergistic effect in treating streams containing high silica in membrane applications. Further, the method as described herein specifically allows for the inhibition of silica scaling on a membrane surface, and allows for a RO or NF system to operate with silica levels of up to 350ppm at a pH of 7.5.

The method comprises providing an antiscalant composition comprising a silica inhibitor and a dispersant; and adding the antiscalant composition to an aqueous stream of an aqueous system. In some embodiments, the antiscalant composition of the disclosed method is a blend of the silica inhibitor and the dispersant. As previously explained, blending may be provided by any conventional blending techniques as described herein.

In some embodiments, the silica inhibitor of the disclosed method comprises an organophosphoric acid, a phosphonate-based compound, or a carboxylic sulphonated copolymer.

In some embodiments, the dispersant of the disclosed method is a sulphonated acrylic acid copolymer or terpolymer. In some embodiments, the sulphonated acrylic acid copolymer or terpolymer comprises repeat units characterized by the formula

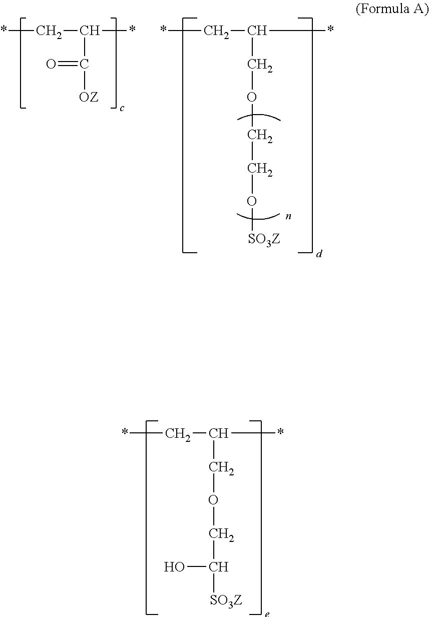

wherein n ranges from about 1-100; and Z is H, Na, K, Ca or NH₄.

In some embodiments, the silica inhibitor as described in the present method comprises 1-hydroxyethylidine-1,1-diphosphonic acid, and the dispersant is a sulphonated/sulfated acrylic acid copolymer, terpolymer, or a sulphonated acrylic acid polymer comprising repeat units characterized by Formula A.

It should be understood that the aqueous system as disclosed herein may be present in, but not limited to, a membrane desalination plant, influent water to an industrial plant, influent water to a beverage plant, or the like. In some embodiments, the aqueous system comprises a reverse osmosis membrane (RO) or a nanofiltration (NF) membrane.

In some embodiments, the aqueous stream comprises a silica content of at least 300 ppm. In other embodiments, the aqueous stream comprises a silica content of about 300 ppm to about 350 ppm. In some embodiments, the aqueous stream comprises a pH of at least 7. In some embodiments, the aqueous stream comprises a pH of about 7.5. In other embodiments, the aqueous stream has a pH of about 7.5 and a silica content of at least 300 ppm.

In some embodiments, the antiscalant composition is added to the aqueous stream in an amount of about 1 ppm to about 100 ppm. In some embodiments, the antiscalant composition is added to the aqueous stream in an amount of about 3 ppm to about 30 ppm.

### EXAMPLES

The present technology will be further described in the following examples, which should be viewed as being illustrative and should not be construed to narrow the scope of the disclosed technology or limit the scope to any particular embodiments.

The antiscalant composition and method as disclosed herein was shown to exhibit a synergistic effect when blended together, and exhibited enhanced performance as compared to other target silica water treatments. Such synergy is believed to be provided by the threshold silica scaling inhibition of the silica inhibitor, and the dispersant's ability to suspend particle dispersions in high silica concentrate feed water.

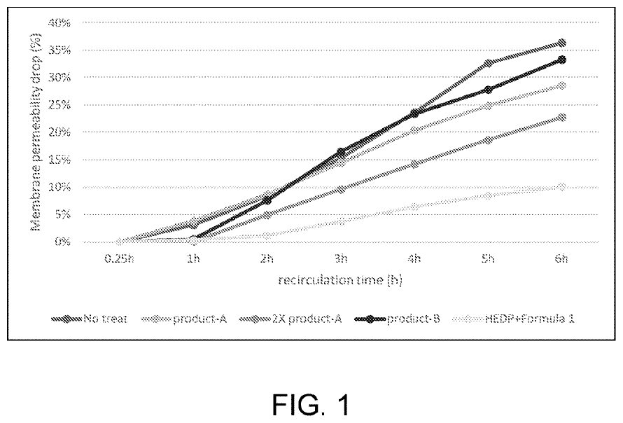

FIGS. 1-4 provide relative data of the silica antiscalant composition performance on water-A treatment (containing 300ppm silica, 161ppm CCPP and pH at 7.5). Product A is phosphonate, and Product B is phosphonate w/ polymer. As shown in FIGS. 1-4, HEDP is the silica inhibitor, and “Formula I” is the sulphonated/sulfated acrylic acid copolymer, terpolymer, or a sulphonated acrylic acid polymer comprising repeat units characterized by Formula A.

The results as shown in FIGS. 1-4 explain that the presently disclosed antiscalant composition and method provides silica inhibition for at least two hours before any such build-up of scale on the membrane surface is exhibited, (i.e. which results in the reduction in permeability). As such, the disclosed antiscalant composition and method is believed to provide improved performance in a field RO/NF system at 300 ppm silica and without acid addition. This is in contrast to the treatment with other commercial products (e.g. Product A and Product B), which demonstrated a reduction in permeability within the first hour and/or exhibited more of a reduction in permeability within two hours.

With reference to FIG. 1, the blend of HEDP and Formula I was shown to outperform the other two commercial products in water-A treatment. The blend of HEDP and Formula I exhibited a much lower membrane permeability drop during 6 hours of recirculation. (Note: there is a 10% permeability drop for HEDP+Formula I observed in FIG. 1 due to it running at an accelerated testing mode condition.)

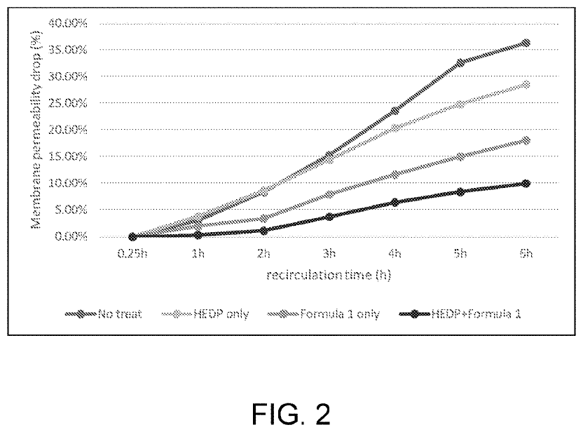

FIG. 2 shows the synergistic effect of the presently disclosed composition. Specifically, the silica antiscalant composition (e.g. the blend of HEDP and Formula I) demonstrated the synergistic effect in water-A treatment as it achieved the lowest membrane permeability drop realized by individual components alone.

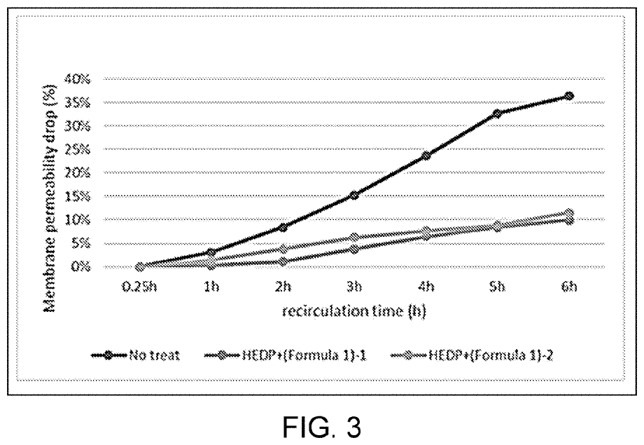

FIG. 3 provides a repeatability test of the disclosed silica antiscalant composition.

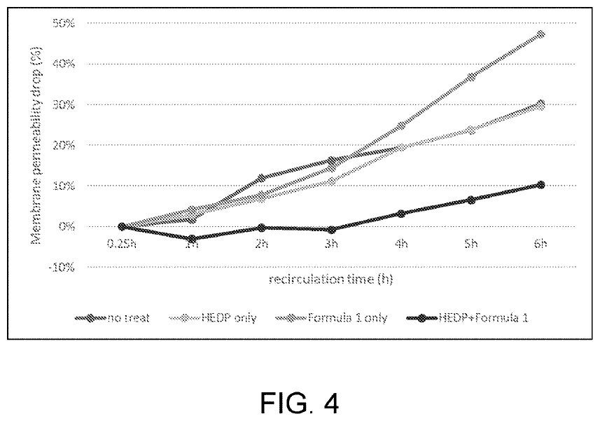

FIG. 4 shows the synergistic effect of the presently disclosed composition on water-H treatment (containing 350ppm silica, 369ppm CCPP and pH at 7.5). Specifically, a similar synergistic effect of the disclosed antiscalant composition was observed for water-H treatment, indicating its silica scaling treatment efficacy is reliable and can be generally applied to different water-chemistry.

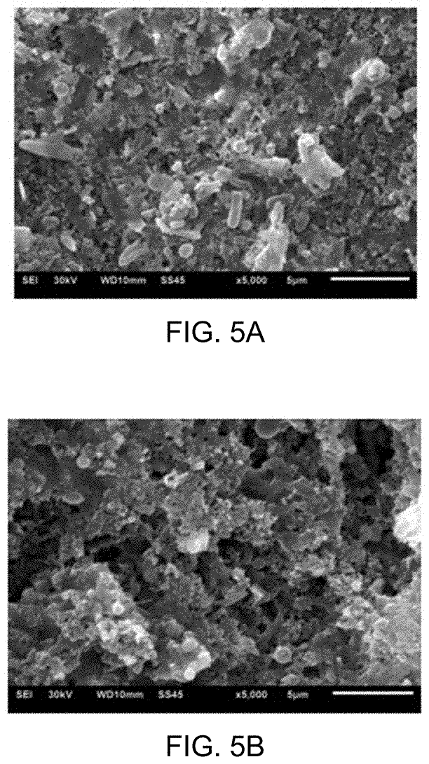

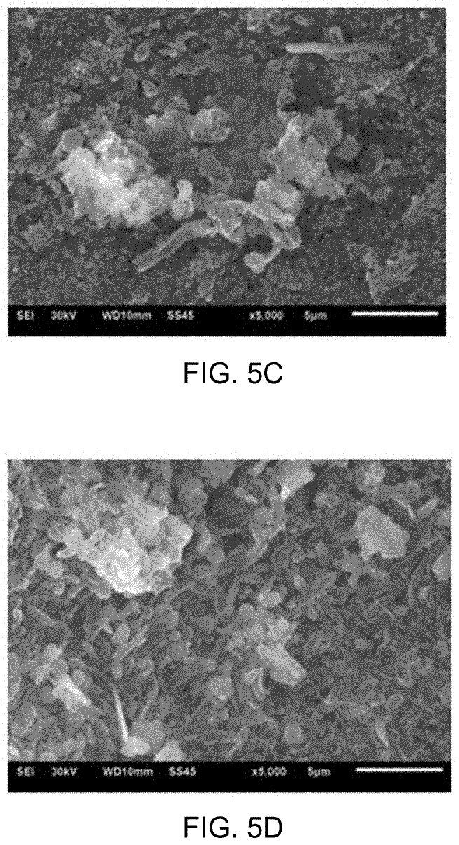

FIGS. 5A-D provide SEM and EDS image results of water-H treatment. As shown in FIGS. 5A-D, a significant reduction of surface deposits was observed when the disclosed antiscalant composition was provided (HEDP and Formula I), which caused a slight increase of membrane resistance and eased membrane permeability decline in 6 hours of recirculation test.

FIG. 5A shows the effect of no treatment and resulted in the presence of 4.9% Si and 8% Ca on the membrane surface. FIG. 5B shows the effect of HEDP treatment only, which resulted in the presence of 8% Si and 0% Ca on the membrane surface. FIG. 5C shows the effect of Formula I treatment only, which resulted in the presence of 12.3% Si and 0.3% Ca on the membrane surface. FIG. 5D shows the effect of the HEDP+Formula I treatment composition, which resulted in the presence of 2.3% Si and 0% Ca on the membrane surface.

While embodiments of the disclosed technology have been described, it should be understood that the present disclosure is not so limited and modifications may be made without departing from the disclosed technology. The scope of the disclosed technology is defined by the appended claims, and all devices, processes, and methods that come within the meaning of the claims, either literally or by equivalence, are intended to be embraced therein.

1. An antiscalant composition, the composition comprising:
a silica inhibitor composition, wherein the silica inhibitor composition comprises an organophosphoric acid, a phosphonate-based compound, or a carboxylic sulphonated copolymer; and
a dispersant composition.

2. (canceled)

3. The composition as recited in claim 1, wherein the silica inhibitor composition is an organophosphoric acid comprising 1-hydroxyethylidine-1,1-diphosphonic acid.

4. The composition as recited in claim 1, wherein the silica inhibitor composition is present at a concentration of about 5-40% actives.

5. The composition as recited in claim 1, wherein the dispersant composition comprises a sulphonated acrylic acid polymer.

6. The composition as recited in claim 5, where the sulphonated acrylic acid polymer comprises repeat units characterized by the formula 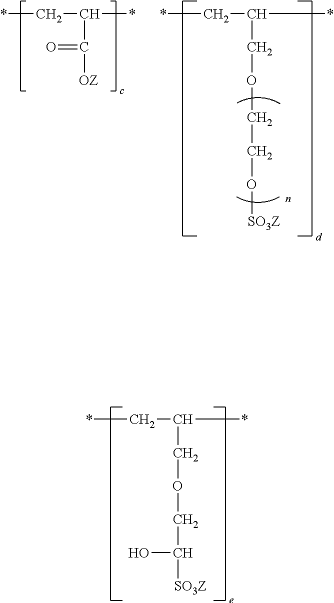
wherein n ranges from about 1-100; and Z is H, Na, K, Ca or NH₄.

7. The composition as recited in claim 6, wherein n is about 1-20.

8. The composition as recited in claim 6, wherein Z may be the same or different in c, d and e.

9. The composition as recited in claim 6, wherein the mole ratio of c:d:e ranges from about 20:10:1 to 1:1:20.

10. The composition as recited in claim 6, wherein the molecular weight of the sulphonated acrylic acid polymer ranges from about 10,000 to about 30,000.

11. The composition as recited in claim 1, wherein the concentration ratio of the silica inhibitor composition to the dispersant composition is about 1:2.

12. (canceled)

13. The composition as recited in claim 1, wherein the silica inhibitor and the dispersant composition are blended together.

14. An antiscalant composition, the composition comprising:
a blend of (i) a silica inhibitor composition, wherein the silica inhibitor composition comprises 1-hydroxyethylidine-1,1-diphosphonic acid; and (ii) a sulphonated/sulfated acrylic acid polymer or terpolymer.

15. The composition as recited in claim 14, wherein the sulphonated acrylic acid polymer or terpolymer comprises repeat units characterized by the formula 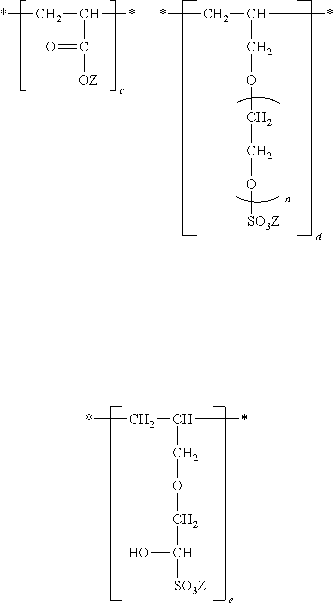
wherein n ranges from about 1-100; and Z is H, Na, K, Ca or NH₄.

16. The composition as recited in claim 14, wherein the silica inhibitor and the dispersant composition are blended together at about 25° C.

17. A method for inhibiting scale formation in a membrane system, the method comprising:
providing an antiscalant composition, the antiscalant composition comprising a silica inhibitor and a dispersant; and
adding the antiscalant composition to an aqueous stream of an aqueous system.

18. The method as recited in claim 17, wherein the silica inhibitor comprises an organophosphoric acid, a phosphonate-based compound, or a carboxylic sulphonated copolymer.

19. The method as recited in claim 17, wherein the dispersant is a sulphonated acrylic acid polymer or terpolymer.

20. The method as recited in claim 17, wherein the antiscalant composition is a blend of the silica inhibitor and the dispersant.

21. The method as recited in claim 20, wherein the silica inhibitor comprises 1-hydroxyethylidine-1,1-diphosphonic acid, and the dispersant is a sulphonated/sulfated acrylic acid polymer or terpolymer.

22. The method as recited in claim 19, wherein the sulphonated acrylic acid polymer or terpolymer comprises repeat units characterized by the formula 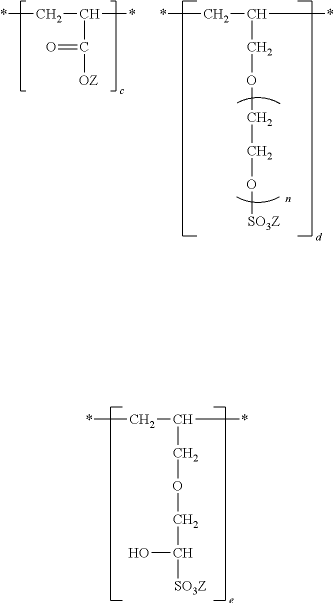
wherein n ranges from about 1-100; and Z is H, Na, K, Ca or NH₄.

23. The method as recited in claim 17, wherein the aqueous stream comprises a silica content of at least 300 ppm.

24. (canceled)

25. The method as recited in claim 17, wherein the aqueous stream comprises a pH of at least 7.

26-28. (canceled)

29. The method as recited in claim 17, wherein the antiscalant composition is added to the aqueous stream in an amount of about 1 ppm to about 100 ppm.

30. (canceled)
